(19) United States
(12) Patent Application Publication
(10) Pub. No.: US 2020/0101019 A1
(43) Pub. Date: Apr. 2, 2020

(54) USE OF LIPOSOMES FOR TREATMENT OF CHRONIC VIRAL HEPATITIS B

(71) Applicant: JIANGSU MENDEL GENE TECHNOLOGY CO., LTD, Jiangsu (CN)

(72) Inventor: Yuzhang Wu, Chongqing (CN)

(21) Appl. No.: 16/495,164

(22) PCT Filed: Mar. 16, 2018
(86) PCT No.: PCT/CN2018/079266

§ 371 Date: Sep. 18, 2019

(30) Foreign Application Priority Data
Mar. 28, 2017 (CN) 201710192607.8

(51) Int. Cl. A61K 9/19; A61K 31/355; A61K 47/42; A61K 47/24

(52) U.S. Cl. CPC A61K 9/19; A61K 31/355; A61K 9/0019; A61K 47/24; A61K 47/42

(57) ABSTRACT

The invention relates to medicinal use of liposomes for the treatment of chronic viral hepatitis B, wherein said liposomes are prepared from phospholipid and cholesterol. This invention further relates to uses of said liposomes for the manufacture of medicaments for promoting the seroconversion of Hepatitis B e antibody to positive and the seroconversion of Hepatitis B e antigen to negative in patients with chronic viral hepatitis B, for reducing the hepatitis B virus titer in the peripheral blood serum of patients with chronic viral hepatitis B, for reducing the concentration of glutamic-pyruvic transaminase in the peripheral blood serum of patients with chronic viral hepatitis B, and for maintaining the stability of the T cell receptor repertoire in patients with chronic viral hepatitis B.

### TECHNICAL FIELD

The invention relates to the technical field of liposomes, and in particular to use of liposomes for treatment of chronic viral hepatitis B.

### BACKGROUND OF THE INVENTION

Viral hepatitis B, as a global disease, is a medical disorder that is caused by Hepatitis B virus (HBV) and mainly characterized by inflammatory lesions on the liver and leads to multiple organ dysfunctions. According to the World Health Organization (WHO) report, people worldwide who are living with HBV infection account for approximately 30% of the population; namely, approximately 1.8 billion people worldwide were once infected with HBV, and about 35 million of them are patients with chronic infection characterized by persistent viremia. China belongs to countries with high prevalence of viral hepatitis B in the world, having approximately 120 million HBV carriers and about 30 million patients living with chronic viral hepatitis B. Most viral hepatitis B is mainly transmitted during the perinatal period, outbreaks during the adolescence and deteriorates in the young manhood. Thus, it mainly endangers young adults. After infection, many patients will recover after natural disease progression, while some patients will undergo delayed, prolonged duration of the disease and may go on to develop cirrhosis or liver cancer. According to the statistics, at least 500,000 patients with chronic infection die of cirrhosis and liver cancer annually throughout the world.

As a hepadnavirus, HBV itself cannot directly cause damage to liver cells. The pathogenesis and clinical outcomes of HBV infection depend on the immune mechanisms. In the cellular immune response, CD8+ cytotoxic T lymphocytes (CTLs) play an important role in the control of viral infections. As extremely strong antigen-specific CTLs are detected in patients with acute HBV infection, the vast majority of patients with acute self-limiting infection can eliminate the viruses and will be cured. Unlike patients with acute viral infection, patients with chronic HBV infection have weak function of CTLs in vivo; moreover, the virus-specific CTLs are gradually attenuated from immunodominance to immune vulnerability during the disease progression of persistent infection, in the diversity of T cell (antigen) receptor (TCR) repertoire diversity decreases, and even the diversity of the overall TCR repertoire decreases in some patients.

The T-cell receptor (TCR) is a molecule found on the T-cell surface that can recognize antigen and is also the most critical molecule to elicit T cell immune response. The diverse repertoire of T cell receptors (TCRs) in the periphery is generated through the rearrangement and selection of respective V(D)JC gene segments during T cell development in the thymus, so that the TCRs are capable of responding to various external antigens. The TCR repertoire is the sum of all the CD8+ T cell antigen receptors having diverse functions in the immune system of an individual at a particular time point, in which the response of HBV-specific CTLs determines the final outcome of HBV infection. After HBV infection, the infected patients with high activity of CTLs will eliminate the viruses and then recover, while the infected patients with waning or undetectable activity of CTLs will proceed into chronic persistent infection status and may further develop cirrhosis or/and liver cancer. Therefore, it is possible to treat the chronic HBV infection and prevent HBV-associated diseases such as secondary cirrhosis and liver cancer by overcoming the immunological tolerance of patients with persistent HBV infection, enhancing the activation of HBV-specific CTL response in vivo, and stabilizing and increasing their TCR diversity response.

Liposomes were first discovered by Bangham in 1965. They are artificially-prepared lipoid globules composed of one or more lipoid bilayers similar to cell membrane encapsulating an aqueous medium. The hydrophilic head portion of the lipoid constituting the bilayer forms the inner and outer surfaces of the membrane, while the lipophilic tail portion is located in the middle of the membrane. Such a structure of liposomes allows them to carry a variety of hydrophilic, hydrophobic and amphoteric substances. In medicine, liposomes are widely used as immunological adjuvants and drug carriers to deliver the drugs into the cells mainly by utilizing the property that liposomes may be fused with cell membranes.

At present, the use of liposomes per se as drugs for the treatment of chronic viral hepatitis B has not been reported yet.

### SUMMARY OF THE INVENTION

The invention provides use of liposomes for the treatment of chronic viral hepatitis B.

A first aspect of the invention relates to use of liposomes for the manufacture of a medicament for the treatment of chronic viral hepatitis B, wherein the liposomes are prepared from materials including phospholipid and cholesterol.

In a preferred embodiment, the liposomes are prepared from materials including phospholipid, cholesterol, palmitic acid and/or vitamin E.

In a preferred embodiment, the molar ratio of the components used for preparing the liposomes is phospholipid:cholesterol:palmiticacid:vitamin E=1:(0.1-1):(0-0.15):(0-0.25).

In a preferred embodiment, the phospholipid is soybean phospholipid, preferably soybean lecithin.

In a preferred embodiment, the liposomes are administered by subcutaneous injection.

In a preferred embodiment, the liposomes are in the dosage form of liquid or freeze-dried liposomes, preferably freeze-dried liposomes. The freeze-dried liposomes are freeze-dried by using human serum albumin or povidone K30 as the excipient, preferably by using human serum albumin as the excipient.

A second aspect of the invention relates to use of the liposomes as described in the first aspect of the invention for the manufacture of a medicament for promoting the seroconversion of Hepatitis B e antibody to positive and the seroconversion of Hepatitis B e antigen to negative in patients with chronic viral hepatitis B.

A third aspect of the invention relates to use of the liposomes as described in the first aspect of the invention for the manufacture of a medicament for reducing the hepatitis B virus titer in the peripheral blood serum of patients with chronic viral hepatitis B.

A fourth aspect of the invention relates to use of the liposomes as described in the first aspect of the invention for the manufacture of a medicament for reducing the concentration of glutamic-pyruvic transaminase in the peripheral blood serum of patients with chronic viral hepatitis B.

A fifth aspect of the invention relates to use of the liposomes as described in the first aspect of the invention for the manufacture of a medicament for maintaining stability of the T cell receptor repertoire in patients with chronic viral hepatitis B.

The liposomes of the invention have a phospholipid bilayer membrane consisting of the most commonly used phospholipids (preferably soybean phospholipids) and cholesterol, in which the former is the main lipoid component and the latter plays the role of stabilizing the phospholipid bilayer membrane. In addition, a small amount of palmitic acid and/or vitamin E may be optionally added. The palmitic acid is capable of increasing the amount of negative charges and enhancing the binding ability of the liposomes, and the vitamin E can prevent the oxidative decomposition of the phospholipids. Mannitol and human albumin may be used as protective agents and excipients during freeze-drying process. The phosphate-buffered saline (preferably at pH 6.5) used in the preparation process slows down the hydrolysis of the phospholipids and regulates the osmotic pressure to isotonic.

The invention has the following beneficial effects:

1. The liposomes of the invention can be prepared on large scale by a simple, convenient and easy to operate process, have good stability in vitro, and can meet the requirements of long-term treatment and multiple dosing for patients with chronic diseases.

2. In clinical studies for patients with chronic viral hepatitis B, it has been found that treatment with the liposomes of the invention can effectively promote the seroconversion of Hepatitis B e antibody to positive and the seroconversion of Hepatitis B e antigen to negative in patients with chronic viral hepatitis B, reduce the HBV titer in the patients' peripheral blood and lower the concentration of serum transaminase. The liposomes of the invention achieved therapeutic efficacy higher than that of the current most mainstream drugs useful for treating HBV infections. Meanwhile, the liposomes of the invention as an immunomodulator can effectively maintain the stability of TCR repertoire of CTLs, indicating that the liposomes themselves can be used as an ideal drug for the treatment of chronic viral hepatitis B.

3. In the invention, the liposomes themselves, without carrying any additional drugs, can be used for treating chronic viral hepatitis B, and achieves good effects.

### BRIEF DESCRIPTION OF THE DRAWINGS

In FIG. 1, FIG. 1a illustrates an ultrastructure of the liposomes of Example 1 of the invention as observed by Transmission Electron Microscope (TEM); and FIG. 1b is a graph showing the relationship between the number of liposomes and the number of lipid bilayer membranes in each liposome of Example 1 of the invention as observed by TEM.

FIG. 2 shows the particle size distribution of the liposomes of Example 1 of the invention as measured by a laser particle size analyzer.

In FIG. 3, FIGS. 3A-3C show the changes in T-cell receptor (TCR) repertoires for patients with chronic viral hepatitis B before (0 week) and after (76 weeks) receiving treatment with the liposomes of Example 1 of the invention (hereinafter referred to as the liposome-treated group), for the untreated patients with chronic viral hepatitis B (hereinafter referred to as the untreated group) at 0 and 76 weeks, and for healthy individuals at 0 and 76 weeks, respectively, as quantitatively analyzed by High-Throughput Sequencing (HTS) methods in Example 3 of the invention. A representative case was selected from each group for the correlation comparison of the TCR repertoires at two time points, with one point representing one T cell clone. By comparison, it was found that the TCR repertoire spectrum of the patients in the liposome-treated group who had received treatment with the liposomes of the invention changed (see FIG. 3A). In contrast to the continuous decrease in the TCR repertoire diversity of the patients in the untreated group (see FIG. 3B), the patients in the liposome-treated group maintained or even expanded the individuals' overall TCR repertoire diversity. As compared with the untreated healthy individuals (see FIG. 3C), new high-abundance T-cell clones occurred in the liposome-treated group, which might indicate that the liposomes induced HBV-specific CD8+ cytotoxic T lymphocytes (CTLs) in vivo in those patients.

FIG. 3D shows the correlation comparisons of clonal abundances of the TCR repertoires in the above-mentioned three groups at the two time points. Correlation analysis was performed by using the abundance of each T-cell clone at the two time points to obtain the correlation coefficients. The correlation coefficients are statistical indicators that are used to reflect the degree of correlation between the variables. When comparing the correlation coefficients of the TCR repertoires at the two time points, the variables refer to the expression levels of each individual T cell clone in the two TCR repertoires. The correlation coefficients of the TCR repertoires between two samples indicate the degree of similarity between the expression levels of each T cell clone in the two samples. The values are ranged from −1 to 1; if the value is closer to 1, it means that the expression levels of each T cell clone in the two samples are closer; and if the value is closer to −1, it means that the change in the expression levels of each T cell clone in the two samples is greater. FIG. 3D further demonstrates that the liposomes themselves can stabilize the TCR repertoire and can maintain a dominant T cell immune response against HBV over a prolonged period of time.

### DETAILED DESCRIPTION OF THE INVENTION

For a better understanding of the invention, hereinafter it will be described in details with reference to the examples. However, it should be appreciated that these examples are merely exemplary embodiments of the invention, not being intended to limit the invention. Any simple modification to the invention under the concept of the invention falls within the protection scope of the invention.

In the invention, all operations were carried out at ambient temperature and under normal pressure unless otherwise indicated.

### Example 1: Preparation of Liposomes and Analysis of their Properties

(1) Preparation, Concentration and Freeze-Drying of the Liposomes

The liposomes of this example were prepared from the following raw materials: soy lecithin, cholesterol, palmitic acid, vitamin E, mannitol (20% sterile aqueous solution), human serum albumin (a solution with a concentration of 20%), diethyl ether, ethanol and phosphate-buffered saline (at pH 6.5, 0.1 mM).

Liposomes were prepared by a process of high-pressure injection in combination with double emulsification. 14.1166 g of soy lecithin, 2.3202 g of cholesterol, 0.4630 g of palmitic acid, and 0.8514 g of vitamin E were added and dissolved in 300 mL of diethyl ether. The solution was filtered through 0.2 μm microporous membrane into an emulsion bottle, and then 300 mL of ethanol was added to form primary emulsion (W/O). The thus obtained emulsion was injected into 14.4 L of water at a temperature of 40° C. and stirred. Then a double emulsion (W/O/W) was formed and liposomes were gradually formed with the evaporation of diethyl ether. Possible free organic solvents in the solution were removed by concentration and dialysis via an ultrafiltration device (the concentration factor of dialysis must be more than 200-fold), to give 0.5 L of liposomes with high concentration, i.e., liposome concentrate. Subsequently, 140 mL of a 20% mannitol aqueous solution, 30 mL of 20% human serum albumin, and 30 mL of phosphate-buffered saline were added into 0.5 L of the liposome concentrate. After thorough mixing, the mixture was separately packaged in a size of 1 mL per bottle. The freeze-drier was pre-cooled to 8° C., and then the sample was placed into it and further cooled to a temperature of −39° C. overnight. Afterwards, the condenser, vacuum pump and heater were started to operate successively to evaporate the organic solvent and moisture in the sample, and finally white porous blocks were obtained. The white porous blocks were then sterilized and packaged to obtain the finished products.

(2) Morphological Observation of a Transmission Electron Microscope (TEM)

The freeze-dried liposome products as prepared in step (1) were re-suspended in 1 mL of sterile water for injection and then filtered with a 0.22 μm polycarbonate membrane filter to remove the impurities. 100 μL of the liposome solution was collected and diluted with PBS (at pH 6.5, 0.1 mM) to 2 mL. The liposome solution was further re-suspended and thoroughly stirred. The thus prepared sample was pipetted onto the copper mesh support film and air dried. The Transmission Electron Microscope (TEM) was started to operate, and the copper mesh loaded with the sample were placed into the Transmission Electron Microscope (at 160,000× magnification) for observation. The results were shown in FIG. 1. As can be seen from FIG. 1a, the liposomes were present as vesicles with relatively uniform sizes, most of them being less than 100 nm; and as can be seen from FIG. 1b, most liposomes contained 2 bilayer membranes, and few liposomes contained 4 or 6 bilayer membranes.

(3) Particle Size Distribution of the Liposomes Measured by a Laser Particle Size Analyzer

The particle size distribution of the prepared liposomes was measured by using a Malvern ZEN 1690 laser particle size analyzer. The freeze-dried liposome products as prepared in step (1) were re-suspended in 1 mL of sterile water for injection and then filtered with a 0.22 μm polycarbonate membrane filter to remove the impurities. 100 μL of the liposome solution was collected, diluted with PBS (at pH 6.5, 0.1 mM) to 2 mL, and thoroughly stirred. 1.2 mL of the above sample was collected and added to the sample container for measurement.

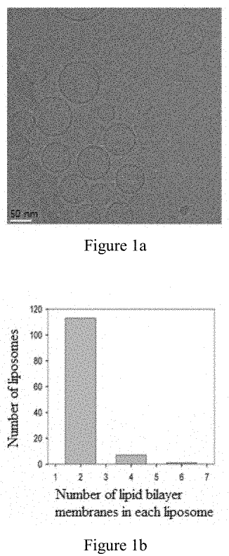

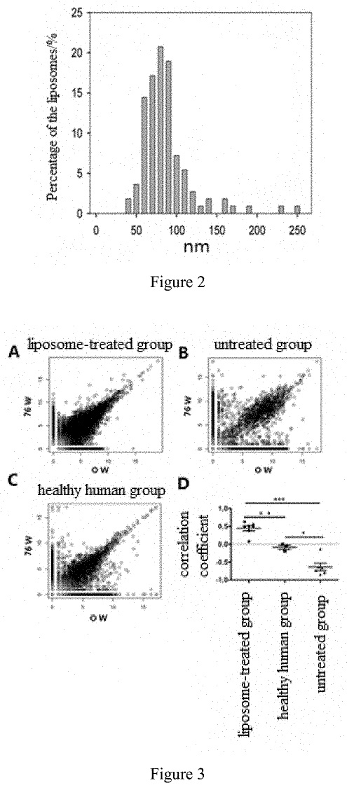

As shown in FIG. 2, the results as analyzed by the laser particle size analyzer were similar to those observed by Transmission Electron Microscope (TEM). It was clear that the particle size distribution of the prepared liposomes was ranged from 30 to 250 nm, mostly from 50 to 100 nm, indicating that the liposomes had particle sizes in nano-scale.

### Example 2: Selection of Excipients for Freeze-Drying of Liposomes

Freeze-Drying of Liposomes and the Selection of Excipients:

Effects of the volume percentage of the liposome concentrate, different excipients (human serum albumin and povidone K30) and their concentrations on the degree of shape shrinkage of the freeze-dried liposomes were compared. The results were shown in Table 1.

TABLE 1
Effects of the volume percentage of the liposome concentrate,
different excipients (human serum albumin and povidone
K30) and their concentrations on the degree of shape
shrinkage of the freeze-dried liposomes

| | Volume percentage* | | Degree of shape |
|---|---|---|---|
| | of the liposome | Excipients and their | shrinkage of the |
| Serial | concentrate (%, | concentrations (%, | liposomes after |
| No. | V/V) | V/V) | freeze-drying |
| 1 | 50 | — | ++++ |
| 2 | 25 | — | +++ |
| 3 | 50 | Povidone K30 (1.0) | +++ |
| 4 | 25 | Povidone K30 (1.0) | ++ |
| 5 | 50 | Povidone K30 (0.5) | +++ |
| 6 | 25 | Povidone K30 (0.5) | +++ |
| 7 | 50 | Human serum | − |
| | | albumin(1.0) | |
| 8 | 25 | Human serum | − |
| | | albumin(1.0) | |
| 9 | 50 | Human serum | + |
| | | albumin(0.5) | |
| 10 | 25 | Human serum | + |
| | | albumin(0.5) | |

Note:
*indicates the volume of the liposome concentrate based on the volume percentage of the emulsion.

As can be seen from Table 1, during the freeze-drying of the liposomes, when the liposome concentrate was 25 to 50% by volume of the volume of the final emulsion, the addition of 1% human serum albumin as the excipient ensured that the shape of the liposomes after freeze-drying did not shrink.

### Example 3: Therapeutic Action of Liposomes in Patients with Chronic Viral Hepatitis B

In this example, the liposome product as prepared in Example 1 was used to treat the selected patients with chronic viral hepatitis B to investigate its therapeutic action and efficacy against chronic viral hepatitis B.

(1) Recruitment of subjects: 119 patients with chronic viral hepatitis B were recruited as the subjects. The specific information of the subjects was as follows:

Years of age

| | | |
|---|---|---|
| Average value (SD) | 27.4 | (7.32) |

| | |
|---|---|
| Medium value | 25.0 |
| Minimum~Maximum value | 17~51 |

Age group, n (%)

| | | |
|---|---|---|
| 17 years old | 1 | (0.8%) |
| 18~45 years old | 115 | (96.6%) |
| 46~65 years old | 3 | (2.5%) |

Gender, n (%)

| | | |
|---|---|---|
| Female | 32 | (26.9) |
| Male | 87 | (73.1%) |

Body Mass Index (BMI), kg/m²

| | | |
|---|---|---|
| Average value (SD) | 22.01 | (2.645) |

| | |
|---|---|
| Medium value | 21.64 |
| Minimum~Maximum value | 16.9~29.4 |

(2) Administration method: 900 μg of the liposome product was administered each time as a subcutaneous injection in the upper arm. The liposome product was dissolved in 3 mL of sterile water, and then administered by subcutaneous injection for 6 times at 0, 4, 8, 12, 20, and 28 weeks of treatment, respectively.

(3) Efficacy evaluation: after the patients with chronic hepatitis B had undergone treatment with the liposomes of Example 1 of the invention, their peripheral blood samples were collected at 12, 28, 32, 40, 52, 64, and 76 weeks, respectively. HBeAg/anti-HBe seroconversion rate, serum HBV titer and the concentration of serum alanine aminotransferase (ALT) were measured to evaluate the efficacy of the treatment with liposomes.

\textcircled{1} Increase of Seroconversion Rate Occurred in Patients Treated with the Liposomes

At the end of the study, 24 of the 119 subjects in the liposome-treated group undergone HBeAg/anti-HBe seroconversion (seroconversion of HBeAg to negative and seroconversion of anti-HBe to positive) with the conversion rate of 20.2% (Table 2). According to the data as described in the Practice Guidelines for Treatment of Chronic Hepatitis B approved by the American Association for the Study of Liver Diseases (AASLD) in 2016 (Table 3), the HBeAg/anti-HBe seroconversion rate after treatment with the liposomes of the invention was second only to that of the pegylated interferon, was equivalent to that of Entecavir, better than that of adefovir and lamivudine, and far higher than that of placebo.

TABLE 2
HBeAg/anti-HBe seroconversion rate in patients
after treatment with the liposomes

| Starting time | Number of patients in which the | |
|---|---|---|
| of the study | conversion occurred (conversion rate, %) | |
| At 12 weeks | 2 | (1.7) |
| At 28 weeks | 9 | (7.6) |
| At 32 weeks | 11 | (9.2) |
| At 40 weeks | 13 | (10.9) |
| At 52 weeks | 13 | (10.9) |
| At 64 weeks | 17 | (14.3) |
| At 76 weeks | 24 | (20.2) |

TABLE 3
HBeAg/anti-HBe seroconversion rate
after treatment with other drugs*

| | Lamivu- | | | | Pegylated |
|---|---|---|---|---|---|
| | dine | Placebo | Adefovir | Entecavir | interferon |
| Conversion rate | 16~21% | 4~6% | 12% | 21% | 27~32% |

*Source of data: from AASLD Guidelines for Treatment of Chronic Hepatitis B, Hepatology. 2016; 63(1): 261-83.

\textcircled{2} Decrease of Serum HBV Titer in Patients after Treatment with the Liposomes

The peripheral blood HBV DNA loads of the subjects who had received treatment with the liposomes of Example 1 of the invention gradually decreased with time

(Table 4). By 76 weeks after the initiation of the study (at 48 weeks after the end of treatment), the proportion of subjects with a decrease in HBV DNA load greater than or equal to 2 logarithmic units was 40.3% (48/119), demonstrating that the liposomes of the invention were advantageous for decreasing serum HBV titers of the patients.

TABLE 4
Percentage of patients with a decrease in serum
HBV DNA load greater than or equal to 2 logarithmic
units after treatment with the liposomes

| Starting time | Number of patients with decreased |
|---|---|
| of the study | titer (conversion rate, %) |
| At 12 weeks | 16 (13.4) |
| At 28 weeks | 29 (24.4) |
| At 32 weeks | 29 (24.4) |
| At 40 weeks | 31 (26.1) |
| At 52 weeks | 28 (23.5) |
| At 64 weeks | 37 (31.1) |
| At 76 weeks | 48 (40.3) |

\textcircled{3} Higher Normalization Rate of Serum ALT Levels in Patients after Treatment with the Liposomes

The Alanine aminotransferase (ALT) in serum is released by liver cells. When the liver cells are damaged, they release more ALT, resulting in higher ALT levels in the peripheral blood. The rates (normalization rates) of the ALT levels returning within the normal ranges in the patients who had received treatment with the liposomes of Example 1 of the invention were gradually increased (Table 5), being 6.7% at 4 weeks and being elevated to 34.5% (41/119) at 76 weeks. Therefore, it was demonstrated that the serum ALT levels of the patients were effectively lowered after they had received treatment with the liposomes of the invention, indicating that the liposomes of the invention could reduce the liver damage caused by HBV infection.

TABLE 5
Normalization rate of serum ALT levels
after treatment with the liposomes

| Starting time | Number of patients whose ALT levels were | |
|---|---|---|
| of the study | normalized (normalization rate, %) | |
| At 4 weeks | 8 | (6.7) |
| At 8 weeks | 14 | (11.8) |
| At 12 weeks | 16 | (13.4) |
| At 28 weeks | 24 | (20.2) |
| At 32 weeks | 22 | (18.5) |
| At 40 weeks | 32 | (26.9) |
| At 52 weeks | 26 | (21.8) |
| At 64 weeks | 32 | (26.9) |
| At 76 weeks | 41 | (34.5) |

(3) Comparison of Efficacy

In the comparative experiment, the untreated group and the healthy human group were used as the control group, respectively. Peripheral blood mononuclear cells of the subjects in the liposome-treated group were collected and separated respectively before and after the administration of the liposomes as prepared in Example 1 of the invention to the liposome-treated group. The peripheral blood mononuclear cells of the subjects in the two control groups were also collected and separated respectively at the two time points corresponding to the time points taken before and after the treatment in the liposome-treated group. The peripheral blood mononuclear cells were subject to deep sequencing for TCR repertoire analysis. FIGS. 3A-3C showed changes in T-cell receptor (TCR) repertoires of the liposome-treated group, the untreated group and the healthy human group at the two time points, respectively. In the liposome-treated group, whether the liposomes enabled long-term survival of the T cells was observed by monitoring the TCR repertoire before treatment with the liposomes and at 76 weeks after treatment with the liposomes. Correlation analysis was conducted based on the abundance at the time points before and after cloning of the respective T cells to obtain the relevant coefficients. As can be seen from FIG. 3D, the liposomes themselves stabilized the TCR repertoire and maintained dominant T cell immune response against HBV for a prolonged period of time, indicating that they played an important role in the treatment of chronic viral hepatitis B infection.

In summary, the present inventors have found that liposomes themselves had good efficacy in the treatment of chronic viral hepatitis B. Such an efficacy was beyond the expectation of those skilled in the art. The inventors presumed that the reasons why the liposomes of the invention were useful for treating chronic viral hepatitis B might be as follows:

First, the lipid components, such as lecithin, cephalin, phosphatidylinositol, and hemolysis lecithin, contained in the liposome had immunomodulatory effects and were beneficial for the treatment of chronic viral hepatitis B. Second, the antigen, e.g., surface antigen, per se of hepatitis B virus contained a large amount of lipid components, which could fuse with the liposome membrane, thereby being enveloped within the liposomes. Moreover, the liposomes could also fuse with the cell membrane of the antigen-presenting cells and thereby released the antigen carried therewith into the cells, thus presenting the antigen through the MHC class I molecular pathway and contributing to the induction of CTL response.

Although the invention has been described with reference to specific embodiments, those skilled in the art will recognize that the embodiments may be changed or modified without departing from the spirit and scope of the invention. The scope of the invention is defined by the appended claims.

1.-10. (canceled)

11. A method of treating chronic viral hepatitis B in patients in need thereof, comprising the step of administering to the patients an effective amount of liposomes prepared from materials including phospholipid and cholesterol.

12. The method of claim 11, wherein the liposomes are prepared from materials including phospholipid, cholesterol, palmitic acid and/or vitamin E.

13. The method of claim 12, wherein the molar ratio of the components used for preparing the liposomes is phospholipid:cholesterol:palmiticacid:vitamin E=1:(0.1-1):(0-0.15):(0-0.25).

14. The method of claim 11, wherein said phospholipid is soybean phospholipid.

15. The method of claim 11, wherein said liposomes are administered by subcutaneous injection.

16. The method of claim 11, wherein said liposomes are in the dosage form of liquid or freeze-dried liposomes.

17. A method of promoting the seroconversion of Hepatitis B e antibody to positive and the seroconversion of Hepatitis B e antigen to negative in patients with chronic viral hepatitis B, comprising the step of administering to the patients an effective amount of liposomes prepared from materials including phospholipid and cholesterol.

18. The method of claim 17, wherein the liposomes are prepared from materials including phospholipid, cholesterol, palmitic acid and/or vitamin E.

19. The method of claim 18, wherein the molar ratio of the components used for preparing the liposomes is phospholipid:cholesterol:palmiticacid:vitamin E=1:(0.1-1):(0-0.15):(0-0.25).

20. A method of reducing the hepatitis B virus titer in the peripheral blood serum of patients with chronic viral hepatitis B, comprising the step of administering to the patients an effective amount of liposomes prepared from materials including phospholipid and cholesterol.

21. The method of claim 20, wherein the liposomes are prepared from materials including phospholipid, cholesterol, palmitic acid and/or vitamin E.

22. The method of claim 21, wherein the molar ratio of the components used for preparing the liposomes is phospholipid:cholesterol:palmiticacid:vitamin E=1:(0.1-1):(0-0.15):(0-0.25).

23. A method of reducing the concentration of glutamic-pyruvic transaminase in the peripheral blood serum of patients with chronic viral hepatitis B, comprising the step of administering to the patients an effective amount of liposomes prepared from materials including phospholipid and cholesterol.

24. The method of claim 23, wherein the liposomes are prepared from materials including phospholipid, cholesterol, palmitic acid and/or vitamin E.

25. The method of claim 24, wherein the molar ratio of the components used for preparing the liposomes is phospholipid:cholesterol:palmiticacid:vitamin E=1:(0.1-1):(0-0.15):(0-0.25).

26. A method of maintaining the stability of the T cell receptor repertoire in patients with chronic viral hepatitis B, comprising the step of administering to the patients an effective amount of liposomes prepared from materials including phospholipid and cholesterol.

27. The method of claim 26, wherein the liposomes are prepared from materials including phospholipid, cholesterol, palmitic acid and/or vitamin E.

28. The method of claim 27, wherein the molar ratio of the components used for preparing the liposomes is phospholipid:cholesterol:palmiticacid:vitamin E=1:(0.1-1):(0-0.15):(0-0.25).
